(19) United States
(12) Patent Application Publication
(10) Pub. No.: US 2007/0203535 A1
(43) Pub. Date: Aug. 30, 2007

(54) Cochlear implants and apparatus/methods for improving audio signals by use of frequency-amplitude-modulation-encoding (FAME) strategies

(75) Inventors: Fan-Gang Zeng, Irvine, CA (US); Kai-Bao Nie, Irvine, CA (US)

Correspondence Address: CROWELL & MORING LLP;INTELLECTUAL PROPERTY GROUP, P.O. BOX 14300, WASHINGTON, DC, 20044-4300, US

(73) Assignee: The Regents of the University of California, Oakland, CA (US)

(21) Appl. No.: 11/657,978

(22) Filed: Jan. 24, 2007

Related U.S. Application Data
(62) Division of application No. 10/229,397, filed on Aug. 27, 2002, now Pat. No. 7,225,027.

(51) Int. Cl. A61N 1/00; H03G 7/00

(52) U.S. Cl. 607/57; 381/106

(57) ABSTRACT

System and method for using Frequency-Amplitude-Modulation-Encoding (FAME) to improve the quality of sound perception for the cochlear implant users and the compress audio signals so that broadband qualities can be achieved with narrow-band transmission channels. In one embodiment, the FAME strategy extracts essential information (changes in amplitude and frequency) and is able to use a narrow-band capacity to provide broad-band (i.e. high quality) audio signals, which can be used in auditory prostheses and telecommunication.

### CROSS-REFERENCE TO RELATED APPLICATIONS

This application claims the benefit of U.S. Provisional Application No. 60/315,278, filed Aug. 27, 2002, the entire contents of which are hereby incorporated by reference.

### STATEMENT REGARDING FEDERALLY SPONSORED RESEARCH

This invention was made with Government support under Grant RO1-DC-02267-07 awarded by the National Institutes of Health. The Government may have certain rights in this invention.

### FIELD OF THE INVENTION

The present invention relates to apparatus and methods for modifying acoustic signals, and more particularly, the invention relates to apparatus and methods that extract changes in amplitude and changes in frequency from acoustic signals, and use those extracted changes to provide high quality audio signals, which can be used in auditory prostheses and telecommunication devices.

### BACKGROUND

All sounds are characterized by changes in amplitude and frequency. The auditory systems of humans and many mammals are sensitive to changes in amplitude and frequency. In the cochlear implants that have heretofore been available, only amplitude changes are extracted and encoded.

The cochlear implants of the prior art have generally employed two types of sound encoding strategies. In one type, only amplitude modulations are extracted and modulate a fixed rate carrier. see, Wilson et al., Better Speech Recognition With Cochlear Implants, Nature. 1991 Jul. 18; 352(6332):236-8. In the other type, filtered raw analog waveforms (including amplitude, frequency modulations and many other components) are delivered directly to electrodes to stimulate the neurons. see, Eddington et al., Auditory Prostheses Research With Multiple Channel Intracochlear Stimulation In Man, Ann Otol Rhinol Laryngol, 1978, 87 (6 Pt 2), 1-39.

Others have attempted to encode fundamental frequency (Fo) in cochlear implants. see, Geurts L, Wouters J., Coding Of The Fundamental Frequency In Continuous Interleaved Sampling processors for cochlear implants, J Acoust Soc Am. 2001 February; 109(2):713-26; Faulkner A, Rosen S, Smith C., Effects Of The Salience Of Pitch And Periodicity Information On The Intelligibility Of Four-Channel Vocoded Speech: Implications For Cochlear Implants, J Acoust Soc Am. 2000 October; 108(4):1877-87.

In audio compression, there has been some recent research using amplitude and frequency modulations to encode speech. see, Potaminanos, A and Maragos P., Speech Analysis And Synthesis Using An AM-FM Modulation Model, Speech Communication, 1999:28, 195-209 Their studies are generally used to extract and trace frequency modulations at or near the format frequency, which varies by itself and has to be encoded during transmission. The present strategy will extract and code only frequency modulations at a fixed center frequency of a narrow band, which is known a priori in both the coder and the decoder, and needs not to be transmitted.

In cochlear implants, either amplitude modulation (only) or the analog waveform is encoded. One of them provides too little (AM only) while the other provides too much indiscriminable information. In audio coding, the encoding strategy has traditionally been considered from the speech production perspective and little perceptual information except for masking is used.

Although there exists a substantial body of knowledge relating to frequency modulation in basic auditory research, there has been little or no work done to encode frequency modulation in cochlear implants (or any other neural prosthetic devices) and use it in audio compression.

### SUMMARY OF THE INVENTION

The present invention uses Frequency-Amplitude-Modulation-Encoding (FAME) to improve the quality of sound perception for the cochlear implant users and to compress audio signals so that broad-band qualities can be achieved with narrow-band transmission channels.

The FAME strategy extracts essential information (changes in amplitude and frequency) and is able to use a narrow-band capacity to provide broad-band (i.e. high-quality) audio signals, which can be used in auditory prostheses and telecommunication.

In cochlear implants, broad-band audio signals are first divided into narrow bands. Frequency and amplitude modulations are independently extracted from each band, and then processed with filtering and compression to produce frequency and amplitude modulated signals that are adequate for the perceptual capability in implant users or the bandwidth limitation of the transmission channels. The band-specific frequency and amplitude modulations may be used to directly stimulate the electrodes implanted in a person's head or resynthesized to recover the original audio signals.

In audio coding, it is very challenging to encode a 10,000-10,300 Hz signal, but it would be much easier to encode the change (300 Hz) centered at that frequency. Since amplitude and frequency changes are independent and contain time information, the FAME strategy essentially transforms a 3-dimensional (amplitude, frequency, and time) encoding problem into a 2-dimensional problem.

The difference between these fundamental frequency encoding strategies and the present FAME strategy is that only fundamental frequency is used to modulate the carrier across some or all bands in the fundamental frequency encoding strategies, while in the applications of FAME strategy in accordance with this invention, the band-specific frequency modulations (which may or may not carry fundamental frequency information) will be extracted and used to modulate the carrier frequency in the corresponding band.

Frequency-Amplitude-Modulation-Encoding (FAME) strategy is aimed at improving perception of music, tonal-language speech, and speech in multiple-talker backgrounds (“cocktail party effect”). The same strategy can also be used to compress audio signals for all communication purposes including wired, or wireless and internet signal transmission, storage and retrieval of audio information.

### BRIEF DESCRIPTION OF THE FIGURES

FIG. 1 is a flow diagram representing an acoustic simulation of the FAME strategy.

FIG. 2 is a flow diagram sowing a method for implementation of the FAME strategy in a cochlear implant.

FIG. 3 is a flow diagram showing a method for using FAME to encode general audio signals.

FIG. 4 is a flow diagram of a method for processing sound according to the present invention, incorporating a novel algorithm of the present invention.

FIG. 4A is a graph (Amplitude vs. Time) of the original sound of FIG. 4.

FIG. 4B is a 4 channel graphic (Amplitude vs. Time) of the sound of FIG. 4 after the “pre-emphasis” and “4˜24 Butterworth Band Pass Filter steps have been performed.

FIG. 4C is a 4 channel graphic (Amplitude vs. Time) of the AM envelope obtained in the method of FIG. 4.

FIG. 4D is a 4 channel graphic (Frequency vs. Time) of the FM signal that results from application of the FAME algorithm and processing steps of the present invention as shown in FIG. 4.

### DETAILED DESCRIPTION OF PRESENTLY PREFERRED EMBODIMENTS

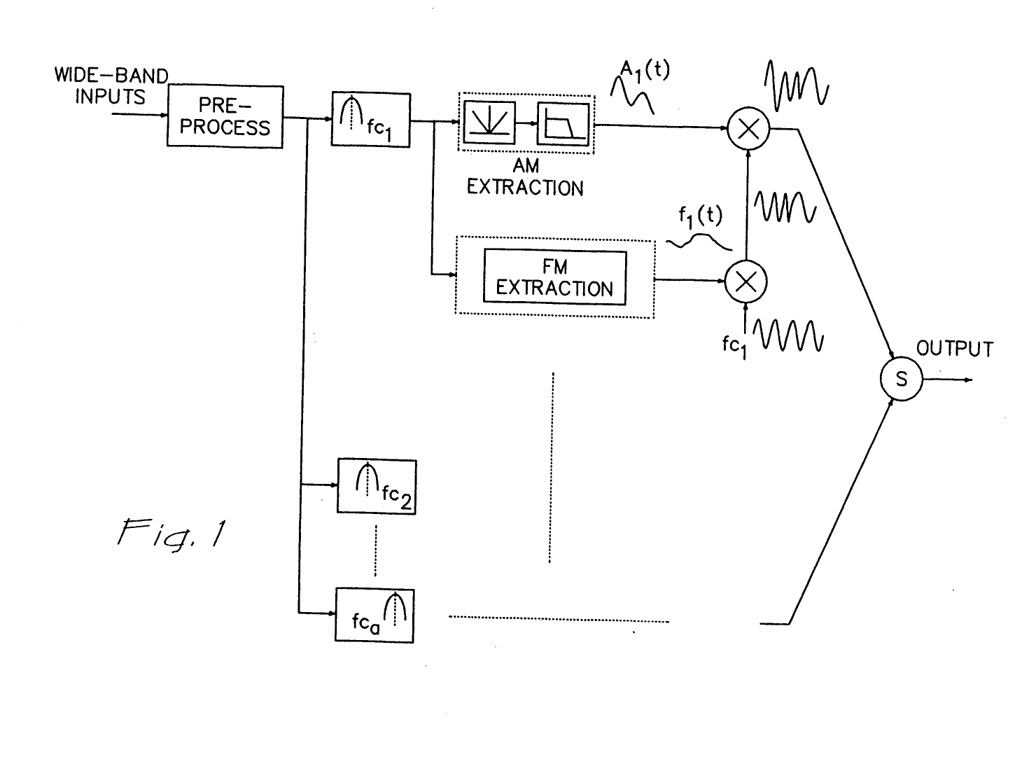

FIG. 1 shows an acoustic simulation of the FAME strategy. A wide-band signal (speech, music or any other audio signals) is first processed to have an ideal bandwidth and spectral shapes, e.g., 20-20000 Hz and spectral flattening for speech sound. The pre-processed audio signal is then filtered into N number of narrow frequency bands. N will be determined based on optimal recognition and compression. The narrow-band signal (only band 1 is demonstrated as an example) will be subject to parallel extraction of amplitude and frequency modulations. Amplitude modulation can be extracted by simple rectification and low-pass filtering as shown in the diagram, or digital Hilbert transfer. Frequency modulation can be extracted by calculating the instantaneous phase angle (frequency) of the fine-structure or zero-crossings of the narrow-band signal. The FM can have a wide range of instantaneous frequency, which will be filtered and/or compressed based on the perceptual evaluations in both normal-hearing and cochlear-implant listeners. In the present implementation, only the 300-Hz FM range is used to modulate a sinusoidal frequency equal to the center frequency of the analysis bandpass filter (fc1). Note that FM changes the frequency of this carrier but not the amplitude of the resulting waveform. The extracted temporal envelope [A1(t)] is then amplitude-modulated to the FM carrier, resulting in a band-specific frequency-amplitude-modulated waveform. These waveforms from all N bands will be summed to produce an acoustic simulation of the FAME strategy.

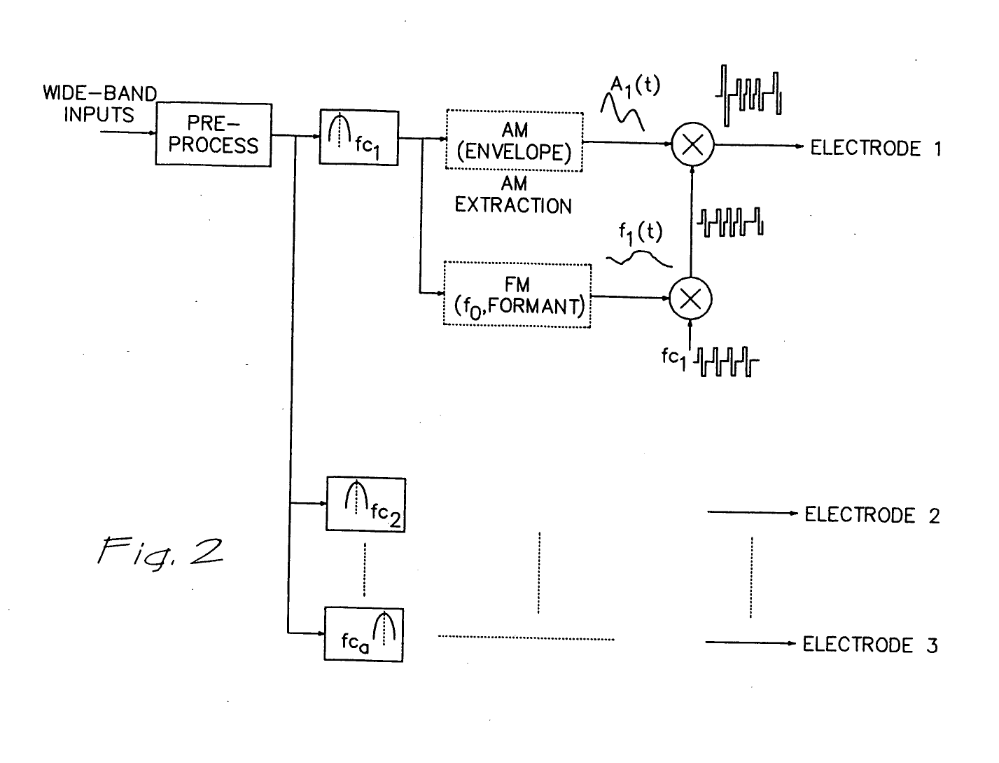

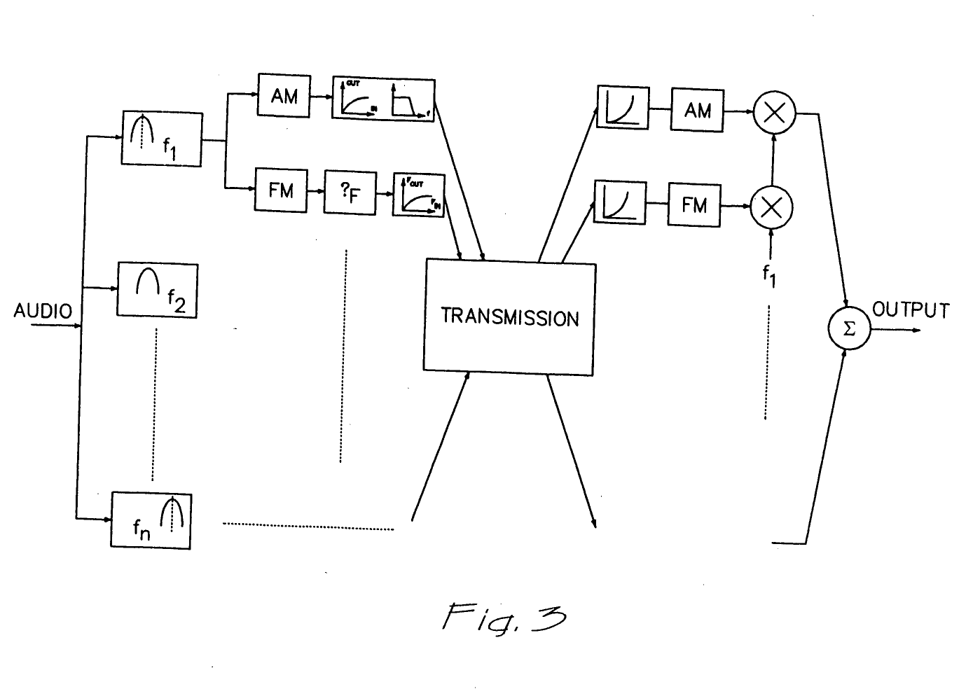

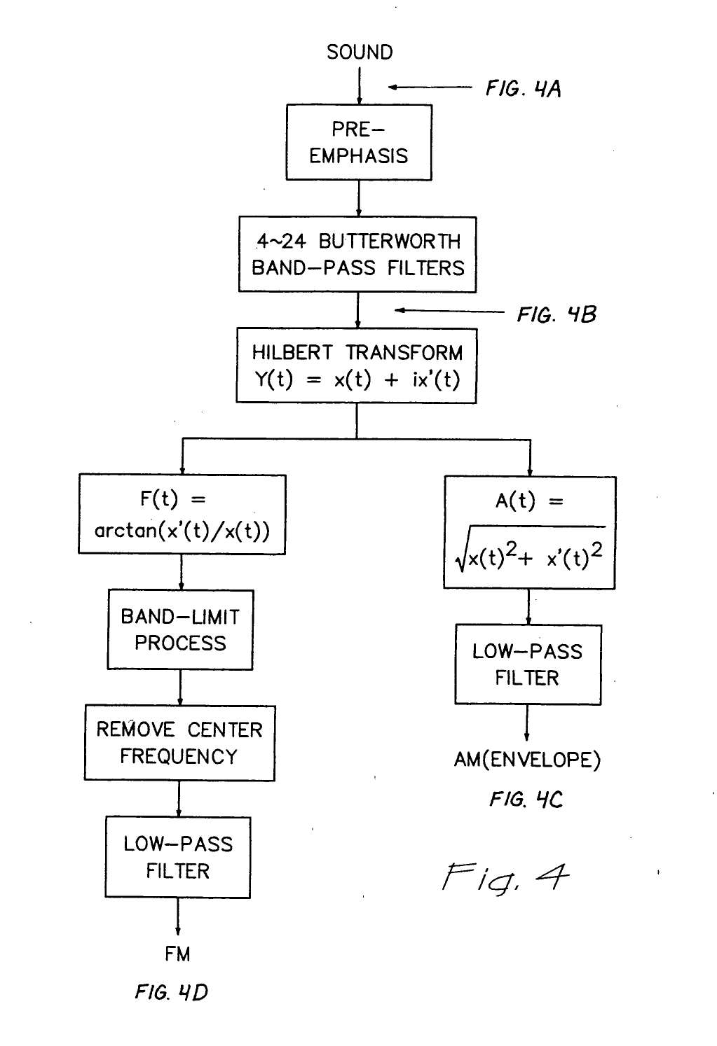

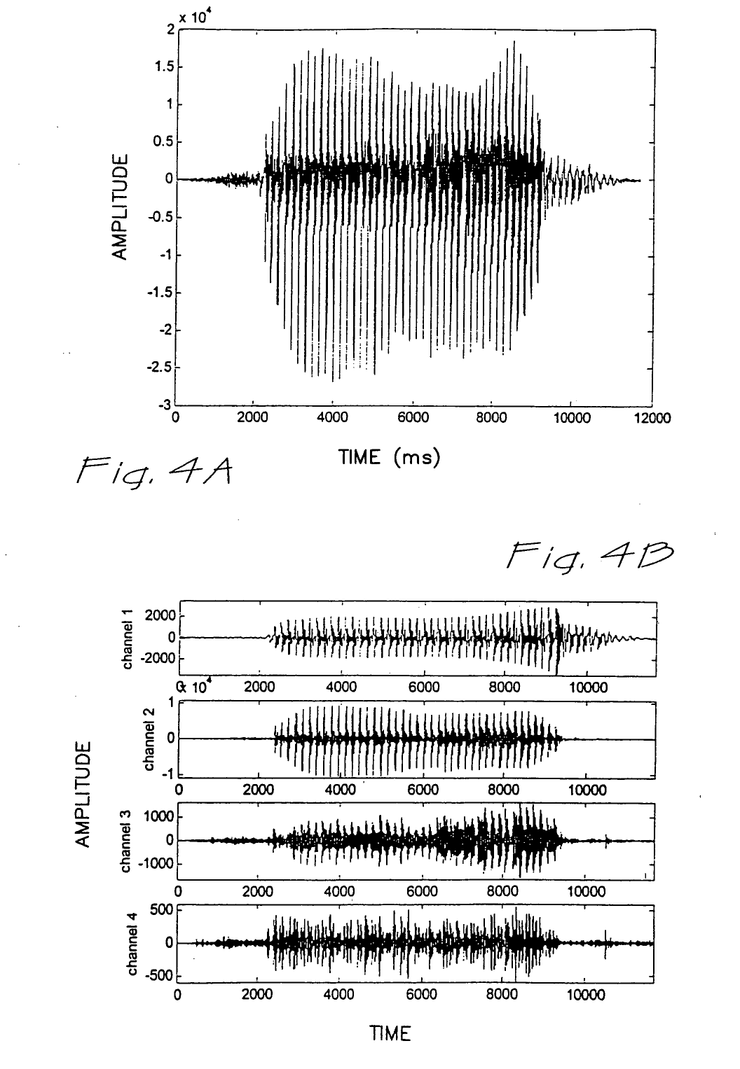

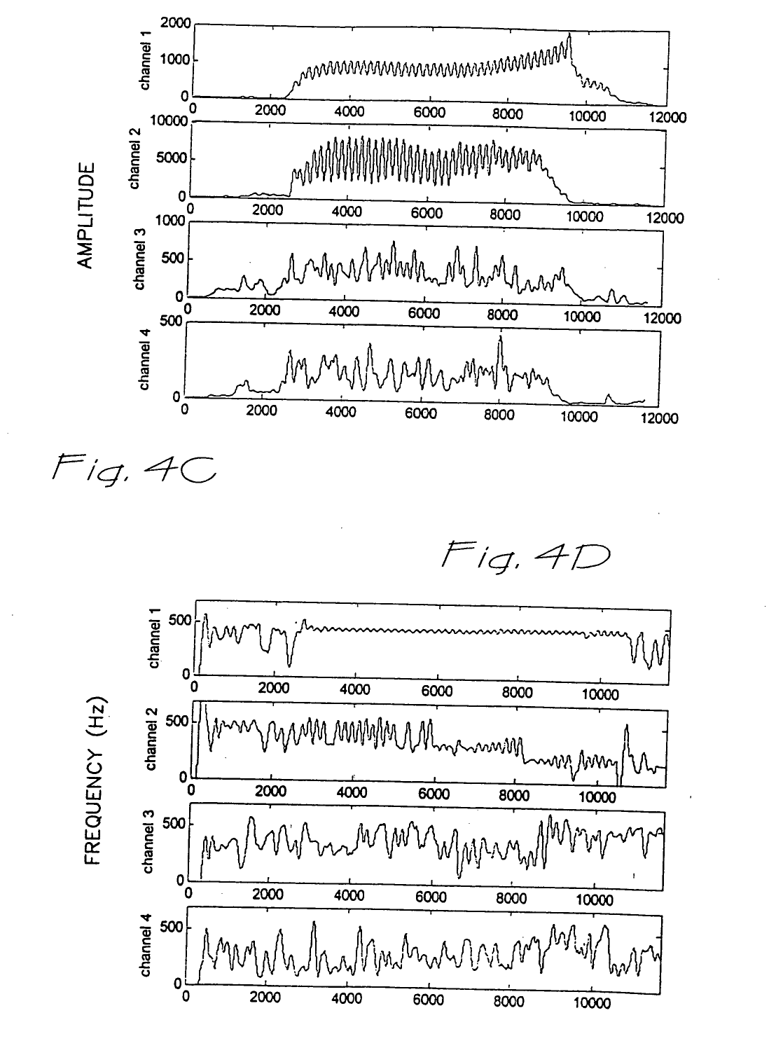

FIG. 2 shows implementation of the FAME strategy in a cochlear implant. All initial processing steps are the same as in the acoustic simulation (FIG. 1) except that in this example, the carrier comprises biphasic pulses. These pulses are first frequency modulated so that inter pulse interval varies according to the frequency modulation (slow-fast-slow) pattern. The FM pulse train will then be amplitude modulated as in the case of the present cochlear implants. Because the perceived place pitch is dominantly coded by the electrode position in the cochlea, the center frequency of the carrier can be either the center frequency of the narrow-band (fcn) or a fixed-rate (e.g., 1000 Hz) pulse train. Alternatively, only FM will be amplitude-modulated to produce final frequency-amplitude-modulated pulses. To avoid the pulse overlap between electrodes, the exact position of the pulses will be varied to result in non-simultaneous stimulation across electrodes. An algorithm will be developed to minimize the change in FM due to the small changes in pulse position within each electrode channel as well as across all channels. An example of one such algorithm is shown in the flow diagram of FIG. 4.

FIG. 3 shows that FAME can be used to encode general audio signals. Band-specific FM and AM will be extracted and compressed for encoded transmission over wired or wireless channels. Because the center frequencies are known on both coding and decoding sides, they need not to be transmitted. The transmitted FM and AM will be restored and synthesized to recover the original audio signal. For each channel, the AM would require 200 bits/sec (8 bits×25 Hz) and the FM would require 300 bits/sec (1 bit zero-crossing×300 Hz), resulting in a total of 500 bits/sec. Because 8-10 channels are likely sufficient to provide high-quality audio signals, a total of 4.8 kbits/sec can be used in a wide range of communication channels.

Cochlear implants and audio compression systems of the present invention (i.e., using the FAME strategy) provide a substantial improvement over the prior art strategy of only coding amplitude modulations. The strategy of coding amplitude modulations, while providing good speech recognition in noise, is not adequate to cope with speech in noise, music perception, and tonal language perception. On the other hand, the analog waveforms theoretically contain all amplitude and frequency modulations, but information regarding these modulations is not accessible to implant users in an unprocessed fashion. Thus, the application of FAME strategies to cochlear implants and audio signals is a significant and inventive improvement.

FIGS. 4-4D show an example of a method of the present invention, wherein a sound (FIG. 4A) is processed to provide an AM (envelope) signal (FIG. 4C) and an FM signal (FIG. 4D) that is obtained by application of FAME strategy using a FAME algorithm in accordance with the present invention.

1. A method for improving sound quality of an analog acoustic signal that is digitally processed, the method comprising the steps of:
a) extracting amplitude modulations and frequency modulations of at least one narrow band of the analog acoustic signal; and
b) filtering and compressing the modulations extracted in step (a) to produce amplitude modulated and frequency modulated acoustic signals that are digitally processed to provide an acoustic signal similar to the analog acoustic signal.

2. The method of claim 1, wherein the method is used to improve sound quality perceived by a person having a cochlear implant, and the method further comprises a step of:
c) stimulating electrodes of the cochlear implant with band-specific frequency and amplitude modulations.

3. The method of claim 1, wherein the method is used to recover broad-band qualities of the analog acoustic signal from a narrow-band transmission of the amplitude and frequency modulations, and the method further comprises a step of:
c) resynthesizing the amplitude and frequency modulations to generate an acoustic signal perceptually similar to the analog acoustic signal.

4. The method of claim 1, further comprising a step of:
dividing the analog acoustic signal into at least one narrow-band acoustic signal.

5. The method of claim 1, wherein the step of extracting the amplitude modulation comprises a step of rectifying and low-pass filtering the narrow band of the analog acoustic signal.

6. The method of claim 1, wherein the step of extracting the frequency modulation comprises a step of calculating an instantaneous phase angle of the narrow band of the analog acoustic signal at a region where the amplitude of the acoustic signal is approximately zero.

7. The method of claim 1, wherein step (b) comprises a step of modulating the amplitude of an extracted temporal envelope from step (a) to a frequency modulated carrier to create a band-specific frequency-amplitude modulated waveform.

8. The method of claim 7, wherein the step of modulating the amplitude of the extracted temporal envelope comprises a step of calculating a square root of the sum of a square of a first amplitude at a first time point and a square of a second amplitude at a second time point.

9. The method of claim 7, further comprising a step of summing the acoustic waveforms from a plurality of narrow bands to produce an acoustic simulation.

10. The method of claim 6, wherein the method comprises measuring a first amplitude at a first time and measuring a second amplitude at a second time, and calculating the arc tangent of the quotient of the second amplitude and the first amplitude.

11-15. (canceled)

16. An audio signal compression system, comprising:
at least one transmitter structured to receive an audio signal;
a plurality of data communication channels;
at least one receiver in communication with the at least one transmitter over the plurality of data communication channels; and
an audio signal encoder that encodes audio signals by (a) extracting amplitude and frequency modulations of at least one narrow band of an analog acoustic signal; and (b) filtering and compressing the modulations extracted in step (a) to produce amplitude and frequency modulated acoustic signals that are transmitted over the data communication channels to the at least one receiver.

17. The system of claim 16, wherein the data communication channels are wireless channels.

18. The system of claim 16, wherein the receiver is configured to restore and synthesize the amplitude and frequency modulated signals to generate an audio signal acoustically similar to the audio signal received by the transmitter.

19. The system of claim 16, wherein the transmitter compresses the audio signals so that the signal information is transmitted over the data communication channels at a rate not greater than approximately 5 kbits/second.

20. The system of claim 16, wherein the transmitter transmits the signal information without center frequency information of the audio signal.
